(19) United States
(12) Patent Application Publication
(10) Pub. No.: US 2008/0154239 A1
(43) Pub. Date: Jun. 26, 2008

(54) CATHETER TUBE ELEMENT

(75) Inventors: Gerhard Gielenz, Wetzikon (CH); Eugen Hofmann, Zurich (CH); Bettina Surber, Duebendorf (CH); Markus Wolfer, Thalwill (CH)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 11/963,167

(22) Filed: Dec. 21, 2007

(30) Foreign Application Priority Data
Dec. 22, 2006 (DE) 10 2006 062 187.5

(51) Int. Cl. A61M 25/09

(52) U.S. Cl. 604/523

(57) ABSTRACT

A multilayered catheter tube element (10), preferably for situating a guide wire, comprising a first layer (11) forming the external layer and a second layer (12) at least partially forming the internal layer. The first layer (11) contains at least one plastic or multiple plastics from the group consisting of PEBA, polyamide, and elastomer-modified polyamide and the second layer (12) contains ethylene tetrafluoroethylene. A cost-effective method for producing a multilayered catheter tube element of this type is also disclosed.

### PRIORITY CLAIM

This patent application claims priority to German Patent Application No. 10 2006 062 187.5, filed Dec. 22, 2006, the disclosure of which is incorporated herein by reference in its entirety.

### FIELD

The present disclosure relates to a multilayer catheter tube element, preferably for situating a guide wire, having a first layer forming the external layer and a second layer at least partially forming the internal layer. The present disclosure also relates to a method for producing a catheter tube element of this type.

### BACKGROUND

Tubes or hoses of various diameters made of materials such as plastic, latex, silicone, metal, or glass are referred to as catheters. With the use of catheters, hollow organs such as the bladder, stomach, colon, blood vessels, ear, or heart may be probed, emptied, filled or flushed. So-called interventional catheters, which are used for interventions, such as percutaneous transluminal angioplasty, have a through opening which is intended to receive a guide wire. To expand or reopen a constricted or closed blood vessel, a balloon catheter, i.e., a catheter which has a balloon element on the distal end, is inserted along the guide wire and placed at the point of the vessel which is to be opened or expanded.

For purposes of the present disclosure, a section of a catheter which extends over at least a part of the length of the catheter is referred to as a catheter tube element. The catheter tube element preferably also comprises the section of a balloon catheter in which the balloon is situated.

For interventional catheterization, catheter tube elements have two layers situated one on top of another which are fastened to one another, are frequently used, these layers having different compositions in their mechanical properties.

A catheter described in European Patent Application No. 0 650 740 B1 has an internal layer, which forms the centrally situated through opening and comprises polyethylene. The external layer comprises polyamide and has a balloon for dilation on the distal end which is welded to the external polyamide layer of the catheter tube. Tubing of this type has the disadvantage that the bonding between the layers is weak so that delamination problems frequently arise. The guide wire frequently jams in the longitudinal lumen of the catheter and is, therefore, carried along by the balloon when the balloon is removed. Therefore, the guide wire must be inserted once again into the vessel for another dilation.

Catheter tubes which comprise an external layer made of a hard plastic material such as polyethylene, polypropylene, polycarbonate, polysulfonate, polymethyl methacrylate, or nylon, for example, and an internal layer made of a soft elastomer plastic material such as PVC, silicone resin, or polyurethane, for example, are described in International Patent Publication No. WO 92/11893 A1. Using these materials, a durable and flexible catheter may be produced which has a small buckling radius and a good spring characteristic (elasticity). However, it is disadvantageous that the external layer is poorly weldable so that a balloon may not be fastened or may not be fastened solidly enough to the catheter tube.

The catheter tubes known from International Patent Publication No. WO 91/01772 A1 have two segments lying next to one another. The first, flexible segment comprises a low density polyethylene (LDPE) or silicone. The second, rigid segment comprises polypropylene or a high density polyethylene (HDPE). The rigid segment occupies 70% to 90% of the total length of the tube so that, in this way, a catheter tube element which is too rigid for many applications in this area is provided.

Further known catheter tube elements are composed of an external polyamide layer and an internal layer made of polytetrafluoroethylene (PTFE). A catheter tube element of this type has very good sliding properties but may not be sterilized by radiation because PTFE is damaged by the ionizing radiation (e.g., x-ray or gamma radiation, also electron bombardment) used for radiation sterilization.

### SUMMARY

The present disclosure describes several exemplary embodiments of the present invention.

One aspect of the present disclosure provides a multilayered catheter tube element, suitable for situating a guide wire, the catheter tube element comprising a) a first layer forming an external layer; and b) a second layer at least partially forming an internal layer, wherein the first layer comprises at least one plastic or multiple plastics from the group consisting of polyether block amide (PEBA), polyamide, and elastomer-modified polyamide and the second layer contains ethylene tetrafluoroethylene (ETFE).

One feature of the present disclosure provides a catheter tube element which has very good bending properties and sliding properties, is weldable on its external surface, and allows radiation sterilization.

The present invention provides, in one exemplary embodiment, a multilayered catheter element of the type cited hereinabove, whose first layer contains a plastic or multiple plastics made from the group consisting of polyamide and elastomer-modified polyamide, such as polyether block amide (PEBA), for example, and whose second layer contains ethylene tetrafluoroethylene (ETFE).

One feature of the catheter element according to the present disclosure is that the first layer comprising one or more of the specified materials is weldable so that a balloon may be welded onto the catheter element. In addition, a good bond may be achieved between the plastics of the first layer and ETFE so that delamination problems do not occur. The plastics used in the catheter element according to the present disclosure, in particular, ETFE, may additionally be sterilized by radiation. A further advantage of the material ETFE is that it has good sliding properties, is chemical-resistant, and may be used at temperatures up to 150° C. ETFE is a very stable fluoropolymer and may be applied in layer thicknesses up to 1000 μm (1 mm). The material is additionally a very good electrical insulator and is chemically resistant to almost all media. Moreover, ETFE may be reshaped thermoplastically, in contrast to PTFE.

The internal layer does not have to cover the entire internal surface of the catheter, but rather may also be situated in a network or line structure.

In one exemplary embodiment, a catheter tube element comprises a hollow cylinder, so that the cross-section is formed by a circular ring. In further exemplary embodiments, the catheter tube element may also be shaped differently, for example, the catheter tube element may have a cross-section in the form of an ellipsoidal ring, a rectangular ring, or another polygonal ring having rounded corners. The continuous opening of the catheter tube element is preferably situated approximately centrally. It is also possible to offset the continuous opening of the catheter tube element somewhat out of the center.

A very simple construction which also results in a very small total diameter of the catheter tube element comprises precisely two layers permanently bonded to one another. For a suitable flexibility of the catheter tube element, the first layer has a degree of hardness between approximately 45 and approximately 72 Shore D. The catheter tube element is also designed very simply if the second layer completely comprises ETFE.

To achieve good sliding properties of the catheter tube element, it is sufficient if the internal second layer is implemented only thinly having a layer thickness of a few micrometers (μm) and preferably has a layer thickness of at least 5 μm, especially preferably at least 10 μm. In contrast, the external first layer has a comparatively greater layer thickness in a preferred exemplary embodiment, preferably a layer thickness of at least 50 μm, especially preferably a layer thickness of at least 80 μm, to ensure sufficient stability of the catheter tube element. In other words, it is advantageous if the ratio of the layer thickness of the first layer to the layer thickness of the second layer is at least 3:1, preferably at least 4:1.

In a preferred exemplary embodiment, the layer thicknesses of the first and the second layers are designed approximately equally over the entire length of the catheter tube element and the cross-section. In further exemplary embodiments, the layer thicknesses of the first and/or the second layer may vary over the length of the catheter tube element and/or the cross-section.

In regard to the method for producing a catheter tube element of this type, one embodiment provides a method in which the second layer is extruded in a first step. Subsequently, the first layer is extruded onto the second layer, which is possibly also provided with further layers. This method is simple and cost-effective and allows the production of catheter tube elements in mass-production scale.

In one exemplary embodiment, the second layer or possibly the external further layer is subjected to a corona treatment before the step of extruding the first layer onto its external surface. The adhesion of the first layer onto the layer lying underneath is thus improved.

A further possibility for producing a catheter tube element of this type uses a multistep immersion method. For this purpose, a preferably cylindrical axis, which keeps the internal cavity of the catheter tube element free, is provided. This axis is subsequently immersed one or more times into an ETFE solution to produce the ETFE layer (second layer) in order to obtain the desired thickness of the ETFE layer. Subsequently, the axis provided with the ETFE layer is immersed in a material solution which contains the material of the first layer. This immersion step may also be repeated multiple times if necessary to obtain the desired layer thickness of the first layer. The axis is removed after finishing this step.

### BRIEF DESCRIPTION OF THE DRAWINGS

Various aspects of the present disclosure are described hereinbelow with reference to the accompanying figures.

Further aspects of the present invention result from the following description of an exemplary embodiment on the basis of the figures. All features described and/or shown in the figures form certain aspects of the subject matter of the present invention per se or in any arbitrary combination, independently of their combination in the individual claims or what they refer back to.

FIG. 1 is a perspective side view of an exemplary embodiment of a catheter tube element according to one exemplary embodiment of the present disclosure; and

FIG. 2 is a front face view of the exemplary embodiment shown in FIG. 1.

### DETAILED DESCRIPTION

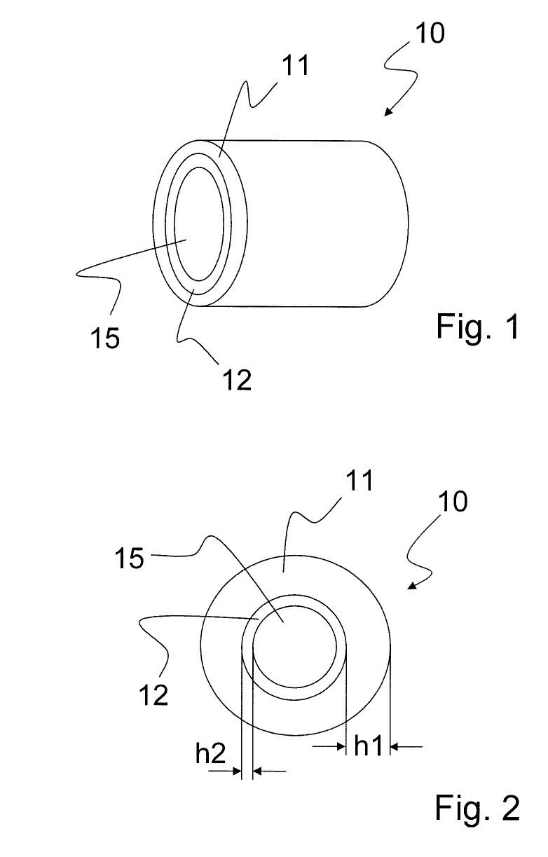

The exemplary embodiment illustrated in FIG. 1 shows a catheter tube element 10 according to the present disclosure, preferably for an interventional catheter, having an external first layer 11 and an internal second layer 12. The catheter tube element according to the present disclosure is designed as a hollow cylinder having an internal lumen 15 formed in the continuous opening, which is intended to situate and preferably to receive a guide wire (not shown). The second layer 12 forms the entire internal wall of the catheter tube element. The first layer 11 forms the entire external mantle surface of the catheter tube element 10. The first layer 11 and the second layer 12 are permanently bonded to one another.

In a further exemplary embodiment, the second layer 12 may also only be situated partially, for example, in a network or strip structure, on the interior side of the first layer 11 and, therefore, only partially form the internal wall of the catheter tube element. In this case, the part of the internal wall not formed by the second layer 12 is implemented by the layer situated below the second layer 12.

The first layer 11 contains one or more plastics from the group consisting of PEBA, polyamide, and elastomer-modified polyamide. The second layer 12 contains ethylene tetrafluoroethylene (ETFE) and completely consists of ETFE in a preferred exemplary embodiment. The first layer 11 completely consists of PEBA or completely consists of polyamide or completely consists of an elastomer-modified polyamide in a preferred exemplary embodiment.

A balloon element (not shown) may be welded to the first layer 11 at a distal end of the catheter tube element.

The layer thickness h1 of the first layer shown in FIG. 2 is at least 50 μm, preferably at least 80 λm. The layer thickness h2 of the second layer 12 is preferably a few micrometers, preferably at least 5 μm, and especially preferably at least 10 μm. The ratio of the layer thicknesses h1/h2 is at least 3:1, preferably at least 4:1. It may be inferred from FIG. 2 that the first layer 11 and the second layer 12 each have identical layer thicknesses over the entire cross-section.

A catheter tube element is provided by the present disclosure which may be sterilized by radiation on one hand and has good bending and sliding properties on the other hand. In addition, the external layer of the catheter tube element is weldable.

All patents, patent applications and publications referred to herein are incorporated by reference in their entirety.

What is claimed is:

1. A multilayered catheter tube element, suitable for situating a guide wire, the catheter tube element comprising:
a) a first layer forming an external layer; and
b) a second layer at least partially forming an internal layer, wherein the first layer comprises at least one plastic or multiple plastics from the group consisting of polyether block amide (PEBA), polyamide, and elastomer-modified polyamide and the second layer contains ethylene tetrafluoroethylene (ETFE).

2. The catheter tube element of claim 1, wherein the catheter tube element comprises a hollow cylinder.

3. The catheter tube element of claim 1, wherein the catheter tube element further comprises two layers.

4. The catheter tube element of claim 1, wherein the first layer comprises a material having a degree of hardness between approximately 45 and approximately 72 Shore D.

5. The catheter tube element of claim 1, wherein the second layer consists essentially of ethylene tetrafluoroethylene.

6. The catheter tube element of claim 1, wherein the second layer has a layer thickness of at least 5 μm.

7. The catheter tube element of claim 1, wherein the first layer has a layer thickness of at least 50 μm.

8. The catheter tube element of claim 1, wherein the ratio of the layer thickness of the first layer to the layer thickness of the second layer is at least 3:1.

9. The catheter tube element of claim 1, wherein the layer thickness of the first layer and the layer thickness of the second layer each remain essentially constant over the entire length of the catheter tube element and the entire cross-section of the catheter tube element.

10. The catheter tube element of claim 1, wherein the catheter tube element is produced using coextrusion or using an immersion method.

11. The catheter tube element of claim 1, wherein the second layer has a layer thickness of at least 10 μm.

12. The catheter tube element of claim 1, wherein the first layer has a layer thickness of at least 80 μm.

13. The catheter tube element of claim 1, wherein the ratio of the layer thickness of the first layer to the layer thickness of the second layer is at least 4:1.
